(19) United States
(12) Patent Application Publication
(10) Pub. No.: US 2025/0059115 A1
(43) Pub. Date: Feb. 20, 2025

(54) STABILISED (HYDRO)HALOGENATED OLEFIN COMPOSITION

(71) Applicant: ARKEMA FRANCE, COLOMBES (FR)

(72) Inventor: Thierry LANNUZEL, Pierre-Benite Cedex (FR)

(73) Assignee: ARKEMA FRANCE, COLOMBES (FR)

(21) Appl. No.: 18/725,073

(22) PCT Filed: Dec. 29, 2022
(86) PCT No.: PCT/FR2022/052518

§ 371 Date: Jun. 27, 2024

(30) Foreign Application Priority Data
Dec. 31, 2021 (FR) FR2114756

(51) Int. Cl. C07C 17/42; C07C 21/18

(52) U.S. Cl. CPC C07C 17/42; C07C 21/18

(57) ABSTRACT

A composition including: at least one (hydro)halogenated olefin having the chemical formula: CX₁X₂═CX₃X₄ (I) where X₁ is selected from F and Cl, and where X₂, X₃ and X₄ are independently selected from H, F and Cl; and, at least one aliphatic alkene, having a boiling point of less than or equal to 80° C. measured at 1013 hPa; wherein the (hydro)halogenated olefin is solely in gaseous form or in the form of a liquid-gas equilibrium.

### TECHNICAL FIELD

The invention relates to the field of hydro(halogenated) olefins with two carbon atoms and notably to their stabilization using a polymerization reaction inhibitor.

### PRIOR ART

In the polymer manufacturing industry, for a large number of ethylene monomers, a major problem relates to the storage and/or transport of these monomers.

Indeed, spontaneous uncontrolled polymerization of these monomers can occur over time, from free radicals which are notably produced by the residual presence of dioxygen, even in trace quantities.

For (hydro)halogenated olefins with two carbon atoms, a specific family of compounds, the terpenes, is commonly used as a stabilizer/inhibitor of polymerization. Among the terpenes, limonene, also called dipentene, is particularly known. U.S. Pat. No. 2,407,405 discloses the stabilization of TFE during storage and handling by virtue of the addition of 0.5% Terpene “B”, which is essentially a mixture of dipentene and terpinolene. Trifluoroethylene is generally sold stabilized with limonene (see Safety Data Sheet for trifluoroethylene sold by Halocarbon—XP002673763).

One of the disadvantages of these organic compounds used as a polymerization inhibitor is their relatively high molecular weight and especially their high boiling point (for example 176° C. for dipentene) in comparison with (hydro)halogenated olefin with two carbon atoms (for example −72° C. for vinyl fluoride, −76° C. for tetrafluoroethylene, −61° C. for trifluoroethylene). Indeed, as (hydro)halogenated olefins with two carbon atoms are generally stored in compressed gas form, optionally liquefied, that is, in gaseous form only or in liquid-vapor equilibrium, this major difference in boiling point and therefore in partial pressure results in a low or even very low concentration of inhibitor in the monomer gas phase.

This amount may prove to be insufficient to protect the monomer in a sustainable and reliable way from spontaneous polymerization.

Another disadvantage of these chemical compounds is their toxicity, which is often high, with regard to human health as well as aquatic life and the environment.

### OBJECTIVES

One objective of the invention is therefore to provide other inhibitors of the polymerization of (hydro)halogenated olefins with two carbon atoms which do not present the disadvantages of the inhibitors described in the prior art.

### DESCRIPTION OF THE INVENTION

The invention relates to a composition comprising:
at least one (hydro)halogenated olefin having the chemical formula:

CX₁X₂═CX₃X₄ (1)
where X₁ is selected from F and Cl, and where
X₂, X₃ and X₄ are independently selected from H, F and Cl; and,
at least one aliphatic alkene, having a boiling point of less than or equal to 80° C. measured at 1013 hPa;
wherein the (hydro)halogenated olefin is solely in gaseous form or in the form of a liquid-gas equilibrium.

The inventors of the present invention surprisingly realized that aliphatic alkenes having a boiling point of less than or equal to 80° C. could play the role of polymerization inhibitor for (hydro)halogenated olefins of formula (I). Additionally, due to their low boiling point, these inhibitors offer the advantage of being able to be introduced at higher gas phase contents than terpenes.

In some embodiments, the (hydro)halogenated olefin of formula (I) is selected from the group consisting of: vinyl chloride, vinyl fluoride, 1,1-dichloroethene, 1,2-dichloroethene, 1,1-difluoroethene, 1,2-difluoroethene, trifluoroethylene, chlorotrifluoroethylene, 1,1-chlorofluoroethene, 1,2-chlorofluoroethene, 1-chloro-2,2-difluoroethylene, tetrafluoroethylene, and mixtures thereof.

In some embodiments, the (hydro)halogenated olefin of formula (I) may essentially comprise an element selected from the group consisting of trifluoroethylene, tetrafluoroethylene, chlorotrifluoroethylene, 1,1-chlorofluoroethene, and mixtures thereof; preferentially from the group consisting of trifluoroethylene, tetrafluoroethylene, and mixtures thereof.

In some particular embodiments, the (hydro)halogenated olefin of formula (I) may essentially comprise trifluoroethylene.

“Essentially comprise” is herein understood to mean that the (hydro)halogenated olefins cited represent more than 95% by weight, preferentially more than 96% by weight, preferentially more than 97% by weight, preferentially still more than 98% by weight and more preferably more than 99% by weight relative to all of the possible (hydro)halogenated olefins.

In some embodiments, the (hydro)halogenated olefin of formula (I) may be selected from the group consisting of trifluoroethylene, tetrafluoroethylene, chlorotrifluoroethylene, 1,1-chlorofluoroethene, and mixtures thereof; preferentially from the group consisting of trifluoroethylene, tetrafluoroethylene, and mixtures thereof.

In some particular embodiments, the (hydro)halogenated olefin of formula (I) may be trifluoroethylene.

In some embodiments, the aliphatic alkene may have a boiling point at most equal to 60° C., and preferentially at most equal to 50° C., measured at 1013 hPa. This has the advantage of being able to introduce the aliphatic alkene at high gas phase contents.

In some embodiments, the aliphatic alkene is a C3 to C6 alkene compound and comprises a single double bond. It may particularly be selected from the group consisting of propene, 1-butene, 2-butene, isobutylene, 1-pentene, 2-pentene, 2-methyl-1-butene, 3-methyl-1-butene, 2-methyl-2-butene, 1-hexene, 2-hexene, 3-hexene, isomers of methylpentene, particularly 4-methyl-1-pentene, isomers of dimethylbutene, cyclopentene, and mixtures thereof.

The aliphatic alkene may particularly be selected from the group consisting of: 1-butene, 2-butene, isobutylene and mixtures thereof.

In some embodiments, the (hydro)halogenated olefin of formula (I) is solely in gaseous form. This form of storage is particularly recommended for certain monomers, such as trifluoroethylene.

In some embodiments, the aliphatic alkene is in liquid-vapor equilibrium. This allows the gas phase to be saturated with aliphatic alkene vapor.

In some embodiments, the (hydro)halogenated olefin of formula (I) represents at least 90%, or at least 91%, or at least 92%, or at least 93%, or at least 94%, or at least 95%, or at least 96%, or at least 97%, or at least 98%, or at least 99% by weight relative to the total composition weight.

In some embodiments, the composition consists essentially of one or more (hydro)halogenated olefins and the aliphatic alkene(s).

The composition may comprise a low content of dioxygen, particularly dioxygen gas. The dioxygen content may be less than or equal to 2500 molar ppm, preferentially less than or equal to 1000 molar ppm, preferentially less than or equal to 500 molar ppm, preferentially still less than or equal to 100 molar ppm, relative to the number of moles of (hydro)halogenated olefin in the gas phase. It is generally desirable for the dioxygen content to be as low as possible in the composition. However, due to the possibility of having a high content of aliphatic alkene in the gas phase allowing polymerization to be inhibited, a certain tolerance can be envisaged in some embodiments. The dioxygen content may be strictly greater than 3 molar ppm, or strictly greater than 50 molar ppm, or strictly greater than 100 ppm, or strictly greater than 300 ppm, or strictly greater than 1000 molar ppm, relative to the number of moles of (hydro)halogenated olefin in the gas phase.

In some embodiments, the composition may comprise more than 100 molar ppm, preferentially more than 250 molar ppm, more preferentially more than 500 molar ppm, more preferentially more than 750 molar ppm, and more preferably more than 1000 molar ppm of said aliphatic alkene in the gas phase, relative to the number of moles of (hydro)halogenated olefin in the gas phase.

In some embodiments, the composition may comprise less than 50 000 molar ppm, preferentially less than 25 000 molar ppm, and more preferably less than 10 000 molar ppm of said aliphatic alkene in the gas phase, relative to the number of moles of (hydro)halogenated olefin in the gas phase. The composition may notably comprise less than 7500 molar ppm, or less than 5000 molar ppm of said aliphatic alkene in the gas phase, relative to the number of moles of (hydro)halogenated olefin in the gas phase.

In some embodiments, the composition may particularly comprise from 1 molar ppm to 100 molar ppm, or from 100 molar ppm to 1000 molar ppm, or from 1000 molar ppm to 2000 molar ppm, or from 2000 molar ppm to 3000 molar ppm, or from 3000 molar ppm to 4000 molar ppm, or from 4000 molar ppm to 5000 molar ppm, or from 5000 molar ppm to 6000 molar ppm, or from 6000 molar ppm to 7000 molar ppm, or from 8000 molar ppm to 9000 molar ppm, or from 9000 molar ppm to 10 000 molar ppm of said at least one aliphatic alkene in the gas phase, relative to the number of moles of (hydro)halogenated olefin in the gas phase.

The invention also relates to the use of an aliphatic alkene such as those mentioned above to stabilize, particularly to avoid any self-polymerization of, a hydro (halogenated) olefin of formula (I), such as those mentioned above.

The above-mentioned compositions may advantageously be used for the storage of hydro (halogenated) olefins of formula (I).

### DETAILED DESCRIPTION OF AN EMBODIMENT

One particular embodiment consists in providing a composition comprising trifluoroethylene and a butene, for example but-1-ene, the butene inhibiting polymerization, particularly self-polymerization, of the trifluoroethylene.

This composition may allow trifluoroethylene to be stored and/or transported whilst particularly avoiding self-polymerization of the monomer.

The trifluoroethylene of the composition may be in in the form of compressed gas, optionally liquefied. It may therefore be solely in gaseous form or in the form of a gas-liquid equilibrium.

The butene of the composition may also be solely in gas form or in the form of a gas-liquid equilibrium.

In some embodiments, the composition comprising trifluoroethylene and butene, capable of being used for storing trifluoroethylene, may be solely in gaseous form.

In some embodiments, the composition comprising trifluoroethylene and butene, capable of being used for storing trifluoroethylene, may comprise a gas phase and a liquid phase, the liquid phase essentially consisting of butene.

Various processes for manufacturing trifluoroethylene are known. Among these processes, the production of trifluoroethylene by hydrogenolysis of chlorotrifluoroethylene is particularly known. Such a process is for example described in application EP 2 819 979 or application EP 2 993 213 (example 1). At the end of the process, a distillation step makes it possible to recover pure or virtually pure trifluoroethylene or trifluoroethylene still containing a small amount of impurities according to the distillation conditions.

In some embodiments, the trifluoroethylene may have a purity greater than or equal to 95.0% (by weight), preferentially greater than or equal to 98.0%, and extremely preferably greater than or equal to 99.0%. In particular embodiments, the trifluoroethylene may have a purity greater than or equal to 99.5%.

For example, document EP 2 993 213 shows the production of a trifluoroethylene with a purity equal to 99.1% comprising, as impurities, other hydrohalogenated olefins, and particularly chlorotrifluoroethylene, isomers of difluoroethylene and isomers of chlorodifluoroethylene, as well as alkanes. No aliphatic alkene, the use of which as an inhibitor of the polymerization of hydro (halogenated) olefins herein claimed, was detected.

The trifluoroethylene thus produced may be stored in a suitable pressure-resistant container. It is stored in the presence of butene in gaseous form with a gas phase content from 1 molar ppm to 100 000 ppm, preferentially from 50 ppm to 75 000 ppm, more preferentially from 100 ppm to 50 000 ppm, more preferentially still from 500 ppm to 25 000 ppm, and extremely preferably from 1000 ppm to 10 000 ppm relative to trifluoroethylene. It may particularly be stored in the presence of butene in gaseous form with a gas phase content from 1000 ppm to 5000 ppm relative to trifluoroethylene.

Prior to the introduction of trifluoroethylene and/or butene and or of the composition comprising trifluoroethylene and butene, the dioxygen content in the container may be reduced to a predetermined threshold by reducing the internal pressure and/or injecting an inert gas.

The composition can be stored under the usual pressure and temperature conditions. The storage temperature may in particular vary from −20° C. to +40° C., or from −10° C. to +35° C. or else from 0° C. to 30° C.

The storage pressure is generally greater than or equal to 1 bar, and less than or equal to 20 bar.

The pressure may in particular be greater than or equal to 1.5 bar or greater than or equal to 2 bar, or greater than or equal to 2.5 bar, or greater than or equal to 3 bar or else greater than or equal to 3.5 bar. A pressure of 3.5 bar or more is beyond the recommended pressure threshold for storing trifluoroethylene comprising less than 1000 ppm of dipentene in the gas phase (see Safety Data Sheet for trifluoroethylene sold by Halocarbon-XP002673763)

The pressure may in particular be less than or equal to 19 bar, or less than or equal to 18 bar, or less than or equal to 17 bar, or less than or equal to 16 bar, or less than or equal to 15 bar. In some embodiments, the storage pressure is between 3.5 bar and 15 bar.

### EXAMPLES

In the experiments performed below, a trifluoroethylene purified to 99.9% and but-1-ene having a purity equal to 99.6% were used.

Chromatographic analysis of trifluoroethylene showed that, among the impurities present, chlorotrifluoroethylene and vinylidene fluoride appear as other aliphatic alkenes. The analysis by chromatography also showed that the trifluoroethylene did not comprise an aliphatic alkene nor, a fortiori, but-1-ene.

### Example 1

An amount of 166 g of trifluoroethylene was reacted with an amount of 280 g of vinylidene fluoride and 17 g of chlorotrifluoroethylene, in the presence of 0.45 g of but-1-ene (i.e. 950 molar ppm relative to the total number of moles of monomers being (hydro)(chloro)fluorinated olefins) in a 4 L reactor containing 3400 g of demineralized water, 1 g of methylhydroxypropyl cellulose and 1.8 g of propyl peroxydicarbonate. The reactor was then brought to a temperature of 44° C. as quickly as possible in order to reach a pressure of 95 bar. Once the temperature of 44° C. was reached, the polymerization reaction only started after an inhibition period equal to 82 minutes, with the reaction being considered as having started after the pressure in the reactor dropped 5 bar.

### Comparative Example

The comparative example was implemented under the same conditions as example 1 except that but-1-ene was not added to the reaction mixture. The reactor was brought to a temperature of 44° C. as quickly as possible and the pressure in the reactor, once the target temperature was reached, was less than 90 bar, which indicates that the polymerization reaction started as the temperature rose in the reactor.

Thus, but-1-ene is an inhibitor of the polymerization of trifluoroethylene.

1. A composition comprising:
at least one (hydro)halogenated olefin having the chemical formula:
CX₁X₂═CX₃X₄ (I)
where X₁ is selected from F and Cl, and where X₂, X₃ and X₄ are independently selected from H, F and Cl; and,
at least one aliphatic alkene, having a boiling point of less than or equal to 80° C. measured at 1013 hPa;
wherein the (hydro)halogenated olefin is solely in gaseous form or in the form of a liquid-gas equilibrium.

2. The composition as claimed in claim 1, wherein the (hydro)halogenated olefin of formula (I) is selected from the group consisting of: vinyl chloride, vinyl fluoride, 1,1-dichloroethene, 1,2-dichloroethene, 1,1-difluoroethene, 1,2-difluoroethene, trifluoroethylene, chlorotrifluoroethylene, 1,1-chlorofluoroethene, 1,2-chlorofluoroethene, 1-chloro-2,2-difluoroethylene, tetrafluoroethylene, and mixtures thereof.

3. The composition as claimed in claim 1, wherein the aliphatic alkene has a boiling point at most equal to 60° C.

4. The composition as claimed in claim 1, wherein the aliphatic alkene is a C3 to C6 alkene compound comprising a single double bond.

5. The composition as claimed in claim 1, wherein the aliphatic alkene is selected from the group consisting of: propene, 1-butene, 2-butene, isobutylene, 1-pentene, 2-pentene, 2-methyl-1-butene, 3-methyl-1-butene, 2-methyl-2-butene, 1-hexene, 2-hexene, 3-hexene, the isomers of methylpentene, notably 4-methyl-1-pentene, the isomers of dimethylbutene, cyclopentene, and mixtures thereof.

6. The composition as claimed in claim 1, wherein the aliphatic alkene is selected from the group consisting of: 1-butene, 2-butene, isobutylene and mixtures thereof.

7. The composition as claimed in claim 1, wherein the (hydro)halogenated olefin is solely in gaseous form.

8. The composition as claimed in claim 1, wherein the aliphatic alkene is in liquid-gas equilibrium.

9. The composition as claimed in claim 1, wherein the (hydro)halogenated olefin represents at least 90% by weight relative to the total composition weight.

10. The composition as claimed in claim 1, comprising more than 100 molar ppm of said aliphatic alkene in the gas phase, relative to the number of moles of (hydro)halogenated olefin in the gas phase.

11. The composition as claimed in claim 1, comprising less than 50 000 molar ppm of said aliphatic alkene in the gas phase, relative to the number of moles of (hydro)halogenated olefin in the gas phase.

12. A method of using at least one aliphatic alkene, having a boiling point of less than or equal to 80° C., measured at 1013 hPa, to stabilize at least one (hydro)halogenated olefin having the chemical formula:
CX₁X₂═CX₃X₄ (I)
where X₁ is selected from F and Cl, and where X₂, X₃ and X₄ are independently selected from H, F and Cl.
